(19) United States
(12) Patent Application Publication
(10) Pub. No.: US 2009/0247717 A1
(43) Pub. Date: Oct. 1, 2009

(54) NEW DINITRO COMPOUND AND ITS CORRESPONDING DIAMINE CONTAINING HETEROCYCLIC THERMAL STABLE COMPOUND AND ORGANOSOLUBLE POLYIMIDE AND POLYIMIDE COPOLYMER

(75) Inventor: Der-Jang Liaw, Taipei (TW)

Correspondence Address: JIANQ CHYUN INTELLECTUAL PROPERTY OFFICE, 7 FLOOR-1, NO. 100, ROOSEVELT ROAD, SECTION 2, TAIPEI, 100, TW

(73) Assignee: National Taiwan University of Science and Technology, Taipei (TW)

(21) Appl. No.: 12/412,335

(22) Filed: Mar. 26, 2009

(30) Foreign Application Priority Data
Mar. 28, 2008 (TW) 97111581

(51) Int. Cl. C08F 26/06; C07D 401/10

(52) U.S. Cl. 526/259; 546/276.7; 528/205

(57) ABSTRACT

A polyimide shown in formula (4) is provided. The polyimide is fabricated by performing a polycondensation reaction with diamine compound shown in formula (2) and various commercial or synthesized dianhydride compounds shown in formula (3) as monomers. In addition, polyimide copolymers are synthesized by various ratios of diamines shown in formulas (2), (7) and (8-1) to (8-7) and a dianhydride compound shown in formula (6). In formulas (3) and (4), Ar represents aromatic groups. In formula (6), X represents aromatic groups or alicyclic groups. In formula (7), n=1 to 10.

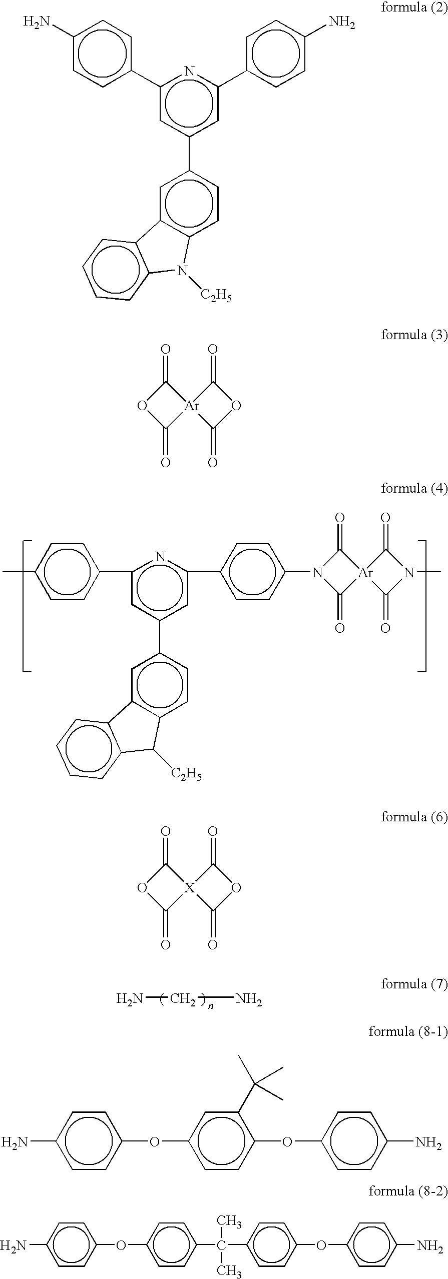

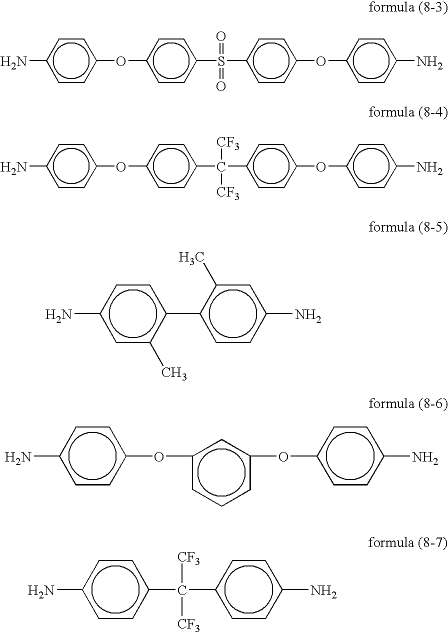

### CROSS-REFERENCE TO RELATED APPLICATION

This application claims the priority benefit of Taiwan application serial no. 97111581, filed Mar. 28, 2008. The entirety of the above-mentioned patent application is hereby incorporated by reference herein and made a part of this specification.

### BACKGROUND OF THE INVENTION

1. Field of the Invention

The present invention generally relates to a dinitro compound, a diamine compound, and a polyimide (PI). In particular, the present invention relates to a new dinitro compound, a new diamine compound derived from the new dinitro compound, and a new PI fabricated by the new diamine compound with various commercial or synthesized dianhydrides, wherein the new diamine compound has a pyridine heterocyclic group and a carbazole-containing electrochromic group.

2. Description of Related Art

Now, PI is one of the important engineering plastics. PI has good thermal stability, resistance to chemical reagents, low dielectric constant, wear resistance, low thermal expansion coefficient, and other excellent properties, such that it is widely applied in automobile industry, semiconductor industry, precision machinery industry, soft printed circuit board, liquid crystal display (LCD), and other electronics industry, and it is a good choice as an electronic material.

Usually, the application of PI in the semiconductor industry is mainly as the material for packaging, coating, adhesive, and other processes. In addition, in the application in the photoelectric industry, PI may be used as an alignment film in LCD devices. For the commercial industrialized PI, the price per kilogram is higher than several million dollars, and recently, most of the raw materials in Taiwan are provided by famous large manufactures in Japan (for example, JSR, CHISSO, Hitachi Chemical, Sumitomo-Chemical, Asahikasei, Ajinomoto, Fine Tech., Ubekosan, NTT, Kanbuchei, New Nippon Steel Company, Central Glass, Sumitomo backlite, Toyota, Toray, Du Pont, Nissan Chemical, Manakku, Mitsui Chemical, Wakayama Seika, and other companies). For the manufacturers producing photoelectric products with the materials, if the academic and the practitioner in Taiwan can provide the production technology and the patent technology transfer, the production quality and the competitive capability of the industry will be greatly improved.

PI is a polycondensation polymer derived from a diamine compound and a dianhydride compound, and the diamine compound is synthesized by dinitro compound. The new PI contains an imide group and can be divided into two categories, namely, rigid aromatic and pyridine groups. The more the benzene rings are, the more improved the thermal endurance, the water proofness, the resistance to chemical reagents, the rubbing resistance, the high glass transition temperature, the hydrophobic property, and other advantages are. Further, the good performance of the electronic product is increased, for example, the alignment film for the photoelectric display.

Aromatic PI has excellent thermal stability, high mechanical strength, high tensile property, high extensibility, and good mechanical properties, so much attention has been paid on the commercial and industrial application thereof

Although the thermal endurance and the mechanical properties of PI are excellent, the problem of poor processibility exists. PI has high melting point or softening point, so it cannot be processed by heating and melting. Further, the solubility is poor, so it cannot be shaped by dissolving in a solvent. Therefore, most of the aromatic PI is difficult to be shaped.

Therefore, it becomes an important issue to prepare PI with good solubility and high thermal stability presently.

### SUMMARY OF THE INVENTION

Accordingly, the present invention is directed to a dinitro compound, a derived diamine compound a PI and copolymers thereof, in which the compound has a pyridine heterocyclic group and carbazole-containing electrochromic group, and the PI compound has good solubility and other characteristics.

The present invention provides a dinitro compound of 4-(9-ethyl-3-carbazole)-2,6-bis(4-nitrophenyl)pyridine (CBNPP), shown in formula (1):

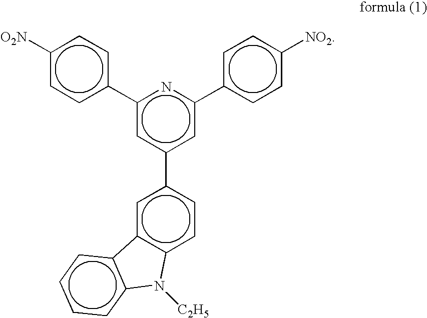

The present invention further provides a diamine compound of 4-(9-ethyl-3-carbazole)-2,6-bis(4-aminophenyl)pyridine (CBAPP), fabricated with the dinitro compound according to claim 1 as monomer, and shown in formula (2):

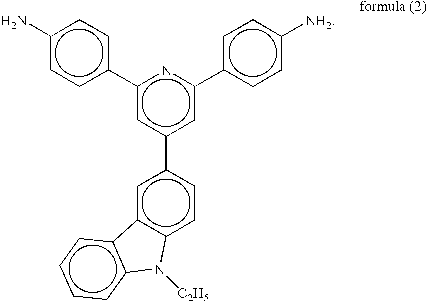

The present invention further provides a polyimide (PI), fabricated by performing a polycondensation reaction with the diamine compound according to claim 2 and a dianhydride compound shown in formula (3) as monomers, and shown in formula (4):

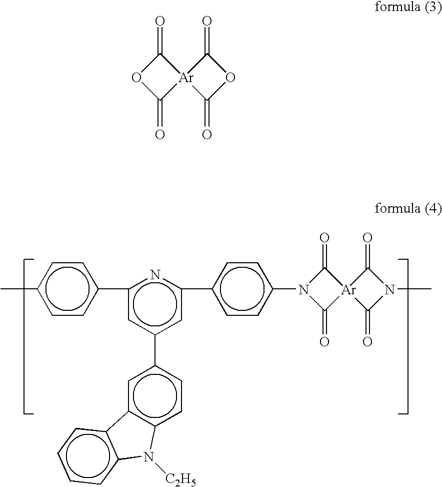

in which Ar in formula (3) and formula (4) represents aromatic groups.

In PI according to an embodiment of the present invention, Ar is a group shown in formulas (5-1), (5-2), (5-3), (5-4), (5-5), (5-6), or (5-7):

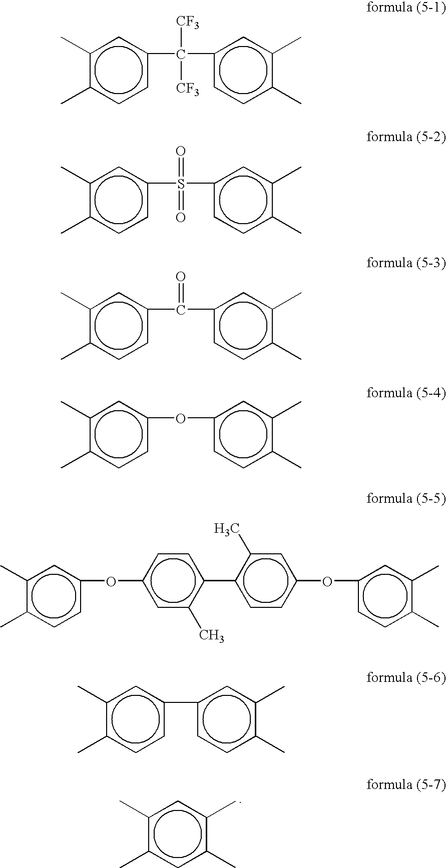

The present invention further provides a polyimide (PI) copolymer, fabricated by performing a polycondensation reaction using the diamine compound according to claim 2, a diamine compound shown in formulas (7), (8-1), (8-2), (8-3), (8-4), (8-5), (8-6) and (8-7) and a dianhydride compound shown in formula (6) as monomers,

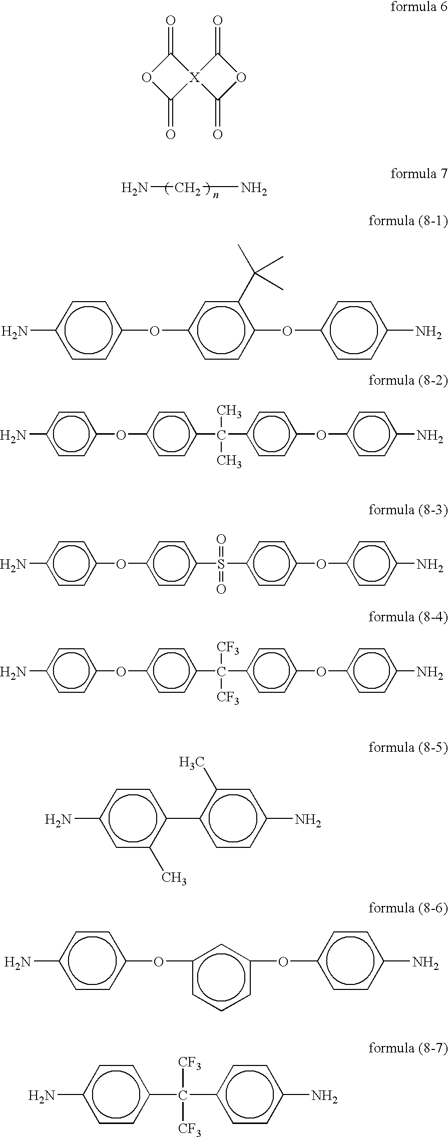

in which X in formula (6) represents aromatic groups or alicyclic groups, and n=1 to 10 in formula (7).

According to an embodiment of the present invention, X in formula (6) is a group shown in formulas (6-1), (6-2), (6-3) or (6-4):

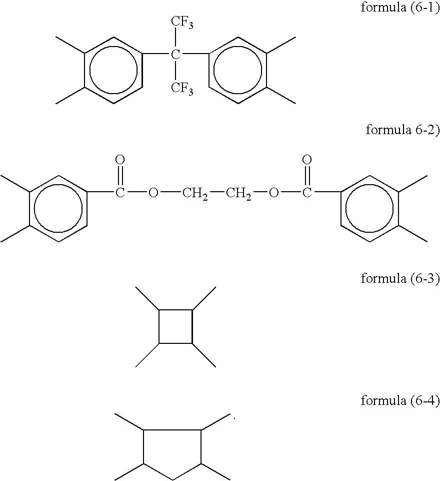

The technical features of the present invention include a new dinitro compound, a new diamine compound derived from the new dinitro compound, a new PI and PI copolymers, which have a pyridine heterocyclic group and carbazole-containing electrochromic group. The new PI has a good solubility, thus the processibility of PI is improved, thereby increasing the application.

### BRIEF DESCRIPTION OF THE DRAWINGS

None.

### DESCRIPTION OF THE EMBODIMENTS

Reference will now be made in detail to the present preferred embodiments of the invention, examples of which are illustrated in the accompanying drawings. Wherever possible, the same reference numbers are used in the drawings and the description to refer to the same or like parts.

Hereinafter, a detailed illustration of a new dinitro compound of the present invention, a new diamine compound fabricated with the dinitro compound, and a new PI fabricated with the diamine compound and a series of dianhydride compounds is given.

### I. Dinitro Compound

The dinitro compound of the present invention is 4-(9-ethyl-3-carbazole)-2,6-bis(4-nitrophenyl)pyridine (CBNPP), shown in formula (1):

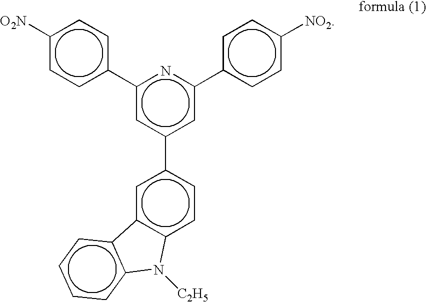

Next, an example is given to illustrate the method of synthesizing CBNPP and the confirmation and analysis of the chemical structure of the fabricated compound.

Firstly, 75 mmol of 9-ethyl-3-carbazole-carboxaldehyde, 150 mmol of 4′-nitroacetophenone, 1.5 mol of ammonium acetate, and 360 ML of acetic acid were placed in a reaction flask, and were reacted at 90° C. for 1 hr, to get a reaction mixture. Then, the reaction mixture was heated to reflux for 72 hr. Next, the cooled reaction mixture was filtered to get the solid part. Then, the obtained solid was recrystallized five times with N,N-dimethylformamide, to get a yellow solid (CBNPP). The melting point of the dinitro compound (CBNPP) is tested to be 280° C., and the yield is 41%.

The synthetic reaction equation of the dinitro compound is shown as follows:

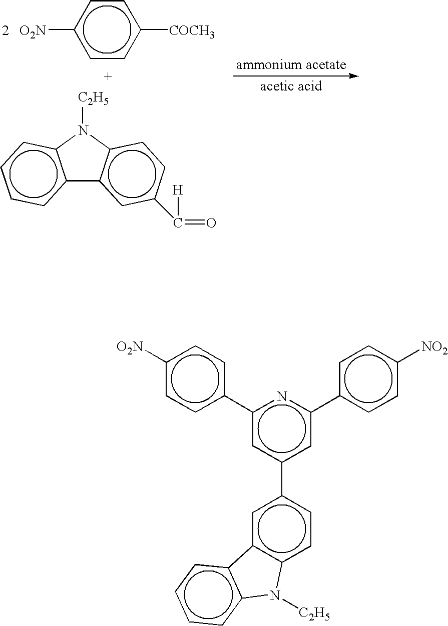

In addition, the obtained dinitro compound (CBNNP) was identified by ¹H-NMR analysis and ¹³C-NMR analysis of nuclear magnetic resonance spectrum (NMR spectrum) and elemental analysis. In the obtained NMR spectrum list, s refers to singlet, d refers to doublet, t refers to triplet, and m refers to multiplet.

¹H-NMR (in which the solvent is DMSO-d₆): δ 8.99(s, 1H), 8.68-8.66(d, 4H), 8.59(s, 2H), 8.41-8.39(d, 4H), 8.35-8.33(d, 1H), 8.25-8.23(d, 1H), 7.80-7.78 (d,1H), 7.66-7.68(d, 1H), 7.54-7.51(t, 1H), 7.31-7.28(t, 1H), 4.55-4.51(m, 2H), 1.39-1.36(t, 3H).

¹³C-NMR(in which the solvent is DMSO-d₆): δ 154.46, 151.08, 147.88, 144.66, 140.41, 140.14, 128.23, 127.03, 126.24, 125.20, 123.84, 122.93, 122.43, 120.76, 119.78, 119.21, 118.65, 109.62, 109.49, 37.15, 13.73.

Elemental Analysis:

Theoretical value: C: 72.36, H: 4.31, N: 10.89, O: 12.44.

Analytic value: C: 72.29, H: 4.32, N: 10.58.

### II. Diamine Compound

The diamine compound of the present invention is 4-(9-Ethyl-3-carbazole)-2,6-bis(4-aminophenyl)pyridine (CBAPP), shown in formula (2):

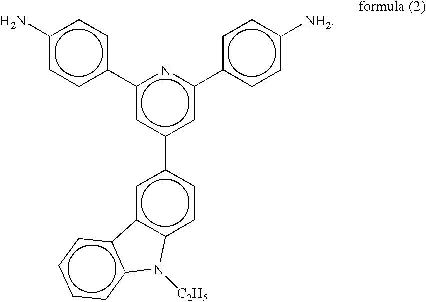

The diamine compound CBAPP is fabricated with the dinitro compound shown in formula (1) as monomer. Particularly, the new diamine compound has a pyridine heterocyclic group shown in formula (2-1) and a carbazole-containing electrochromic group shown in formula (2-2):

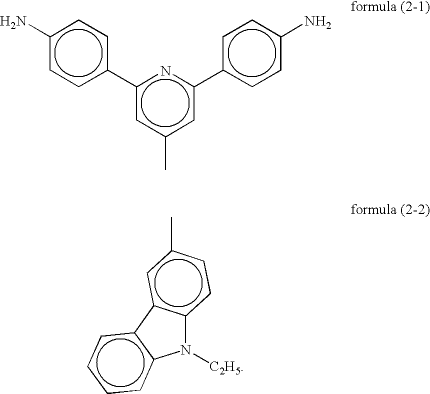

Next, an example is given to illustrate the method of fabricating CBAPP and the confirmation and analysis of the chemical structure of the fabricated compound.

Firstly, 8.6 mmol of the dinitro compound (CBNPP) monomer, 0.15 g of 10% active Pd/C, and 35 mL of ethanol were placed in a reaction flask, and then heated to 90° C., and 20 mL of monohydrate hydrazine (H₂NNH₂.H₂O) was slowly added. After the hydrazine was added, the reaction was performed for 24 hr. After the reaction was finished, the reaction mixture was filtered when it was hot to remove the 10% active Pd/C, so as to get a filtrate. After being cooled and precipitated, the obtained filtrate was filtered again to get the solid part. Then, the obtained solid was recrystallized twice with ethanol, to get a white diamine compound, which was dried in vacuum. The melting point of the diamine compound (CBAPP) is tested to be 125° C., and the yield is 44%.

The synthetic reaction equation of the diamine compound (CBAPP) is shown as follows:

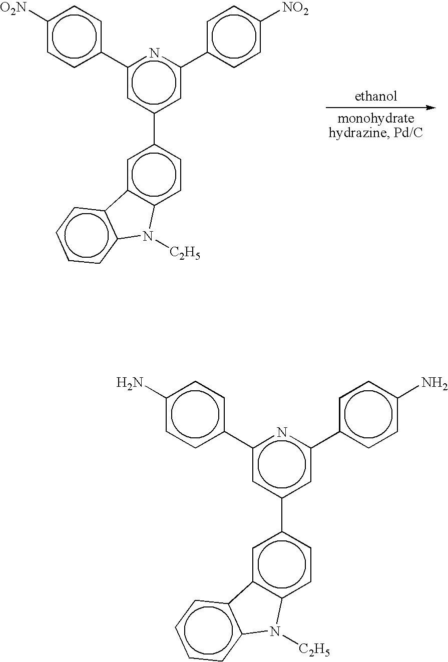

In addition, the obtained diamine compound (CBAPP) was identified by ¹H-NMR analysis and ¹³C-NMR analysis of NMR spectrum and elemental analysis.

¹H-NMR (in which the solvent is DMSO-d₆): δ 8.89(s, 1H), 8.40-8.38(d, 1H), 8.17-8.38(d, 4H), 8.10-8.08(d, 1H), 8.03(s, 2H), 7.71-7.70(d, 1H), 7.60-7.58(d, 1H), 7.49-7.46(t, 1H), 7.28-7.25(t, 1H), 6.81-6.79(d, 4H), 5.44(s, 4H), 4.46-4.42(m, 2H), 1.33-1.31(t, 3H).

¹³C-NMR (in which the solvent is DMSO-d₆): δ 156.52, 149.76, 149.38, 140.93, 139.93, 129.10, 127.78, 126.95, 126.03, 124.81, 122.88, 122.47, 120.86, 119.13, 118.95, 113.72, 112.65, 109.41, 109.24, 37.06, 13.66.

Elemental Analysis:

Theoretical value: C: 81.91, H: 5.77, N: 12.33.

Analytic value: C: 81.84, H: 5.86, N: 12.07.

### III. Polyimide (PI)

PI of the present invention has a structure shown in formula (4), and is fabricated by performing a polycondensation reaction with the diamine compound (CBAPP) shown in formula (2) and the dianhydride compound shown in formula (3) as monomers:

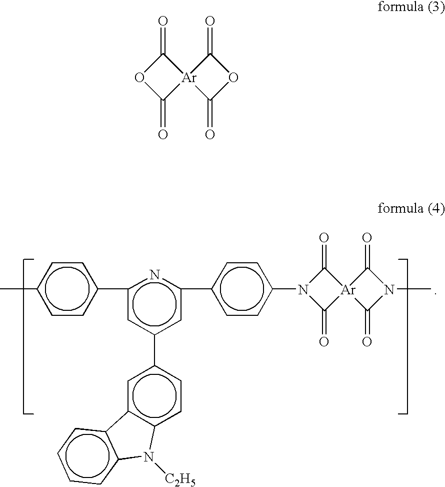

Ar in formula (3) and formula (4) represents aromatic groups. The new PI is derived from the diamine compound (CBAPP) shown in formula (2), so the new PI has the pyridine heterocyclic group shown in formula (2-1) and the carbazole-containing electrochromic group shown in formula (2-2).

In an embodiment, Ar in formula (3) and formula (4) is a group shown in formulas (5-1), (5-2), (5-3), (5-4), (5-5), (5-6), or (5-7), and formula (5-1) is fluorine-containing group, and formula (5-5) is a non-coplanar group:

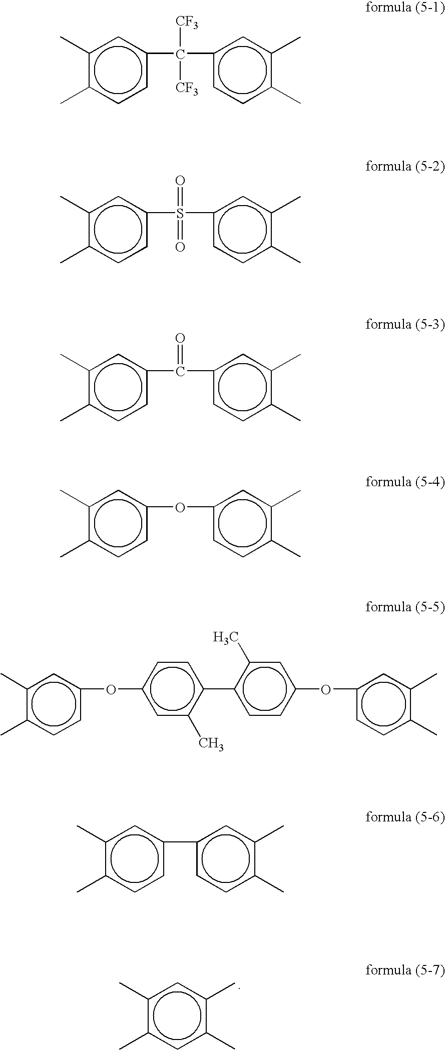

Accordingly, when Ar is a group respectively shown in formulas (5-1), (5-2), (5-3), (5-4), (5-5), (5-6), and (5-7), the dianhydride compound shown in formula (3) is respectively 2,2′-bis(3,4-dicarboxyphenyl)hexafluoropropane dianhydride (6FDA), 3,3′,4,4′-diphenylsulfone tetracarboxylic dianhydride (DSDA), 3,3′,4,4′-benzophenone tetracarboxylic dianhydride (BTDA), 4,4′-oxydiphthalic dianhydride (OPDA), 2,2′-dimethyl-4,4′-bis[4-(3,4-dicarboxyphenoxy)]biphenyl dianhydride, 3,3′,4,4′-biphenyl tetracarboxylic dianhydride (BPDA), pyromellitic dianhydride (PMDA), and the obtained PI is respectively represented by PI-1, PI-2, PI-3, PI-4, PI-5, PI-6, and PI-7.

### IV Polyimide (PI) Copolymer

The dianhydride compound has an excellent thermal property, such as high thermal decomposition temperature and high glass transition temperature (Tg). Further improvement in the film-forming property can be achieved by performing a polycondensation reaction to the diamine compound shown in formula (2) and following formulas (7) and (8-1) to (8-7) in various ratios with the dianhydride compound shown in formula (6), preferably in various molar ratios.

PI copolymer of the present invention is fabricated by performing a polycondensation reaction using the diamine compound shown in formulas (2), formulas (7) and (8-1) to (8-7) and the dianhydride compound shown in formula (6) as monomers in various molar ratios.

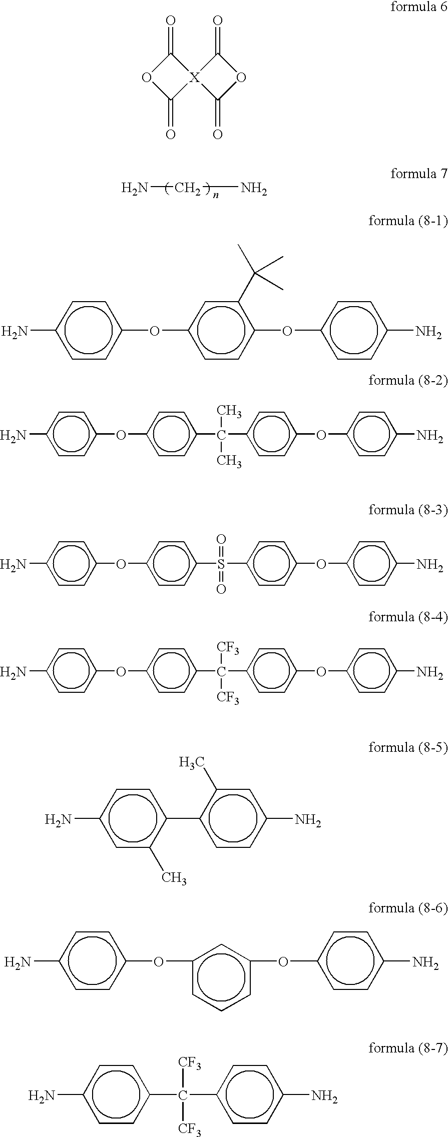

In formula (6), X represents aromatic groups or alicyclic groups. In formula (7), n=1 to 10, preferably n=2 or 6. The new PI copolymer is derived from the diamine compound (CBAPP) shown in formula (2) and the diamine compound shown in formulas (7), (8-1), (8-2), (8-3), (8-4), (8-5), (8-6) and (8-7).

In an embodiment, X in formula (6) can be a group represented by formulas (6-1), (6-2), (6-3) or (6-4) as follows:

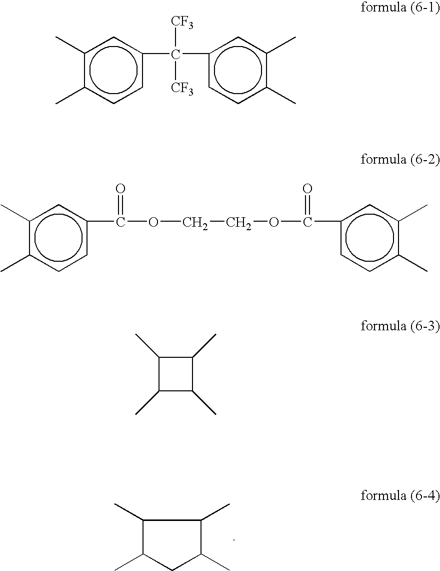

Accordingly, when X is a group respectively shown in formulas (6-1), (6-2), (6-3) and (6-4) respectively, the dianhydride compound shown in formula (6) is respectively 2,2′-bis(3,4-dicarboxyphenyl)hexafluoropropane dianhydride (6FDA), etheylene glycol bis(4-trimellitate-anhydride) (Rica acid), 1,2,3,4-cyclobutane tetracarboxylic dianhydride and 1,2,3,4-cyclopentane tetracarboxylic dianhydride.

Next, the embodiment of fabricating PI and PI copolymers of the present invention is illustrated. Hereinafter, the present invention is described in detail according to a plurality of embodiments but the present invention is not limited to the embodiments.

### [Embodiment 1] Synthesis of PI (CBAPP-6FDA) (PI-1)

1.42 mmol of diamine compound (CBAPP) was dissolved in 3.5 mL of N-methyl-2-pyrrolidinone (NMP). After it was completely dissolved, 1.42 mmol of 6FDA dianhydride monomer was slowly added and reacted for 24 hr to get a viscous poly(amic acid) solution. Then, 0.5 mL of acetic anhydride and 0.5 mL of pyridine were added to the poly(amic acid) solution through a chemical cyclodehydration process, and stirred at room temperature for 1 hr. Then, the reaction mixture was heated to be lower than 100° C. and reacted with stirring for 3 hr. After the reaction was finished and the reaction mixture was cooled, the NMP solution was poured into a great amount of methanol for precipitation, and the polymer was rinsed by methanol and dried in vacuum at 100° C. to get PI (PI-1).

In addition, the chemical structure of PI (PI-1) was confirmed and the characteristics of PI (PI-1) were analyzed as follows.
Relative viscosity: 1.35 in N,N-dimethylacetamide (DMAc) (solution concentration is 0.5 g dL⁻¹, and the measurement temperature is 30° C.).
Solubility: PI-1 is soluble in N-methyl-2-pyrrolidinone (NMP), pyridine, THF, N,N-dimethyl acetamide (DMAc), dimethyl sulfoxide (DMSO), chloroform, and cyclohexanone.
Thermal property: The glass transition temperature is higher than 350° C., the temperature of 10% decomposition is 535° C. in nitrogen, and the temperature of 10% decomposition is 520° C. in air.

Thin film mechanical property: The tensile strength is 61.0 MPa, the elongation is 5%, and the tensile coefficient is 2.0 GPa.

¹H-NMR (in which the solvent is DMSO-d₆): δ 8.72(s, 1H), 8.48-8.47(d, 1H), 8.28-8.26(d, 2H), 8.23-8.22(d, 1H), 8.11-8.10(d, 2H), 8.04(d, 1H), 8.00(s, 1H), 7.95(s, 4H), 7.68-7.67(d, 4H), 7.64-7.62(d, 1H), 7.50-7.49(d, 1H), 7.44-7.43(d, 1H), 7.22-7.21(d, 1H), 4.44(s, 2H), 1.41(s, 3H).

### [Embodiment 2] Synthesis of PI (CBAPP-DSDA) (PI-2)

PI (PI-2) was synthesized in the same manner as that of Embodiment 1, except that 1.23 mmol of diamine compound (CBAPP) was used and the dianhydride monomer was 1.23 mmol of DSDA.

In addition, the characteristics of PI (PI-2) were analyzed as follows. Relative viscosity: 1.20 in DMAc (solution concentration is 0.5 g dL⁻¹, and the measurement temperature is 30° C.).
Solubility: PI-2 is soluble in NMP and DMAc.
Thermal property: The glass transition temperature is higher than 350° C., the temperature of 10% decomposition is 475° C. in nitrogen, and the temperature of 10% decomposition is 465° C. in air.

### [Embodiment 3] Synthesis of PI (CBAPP-BTDA) (PI-3)

PI (PI-3) was synthesized in the same manner as that of Embodiment 1, except that 1.47 mmol of diamine compound (CBAPP) was used and the dianhydride monomer is 1.47 mmol of BTDA.

In addition, the characteristics of PI (PI-3) were analyzed as follows.
Solubility: PI-3 is soluble in NMP, pyridine, THF, DMAc and DMSO.
Thermal property: the glass transition temperature is higher than 350° C.

### [Embodiment 4] Synthesis of PI (CBAPP-ODPA) (PI-4)

PI (PI-4) was synthesized in the same manner as that of Embodiment 1, except that 1.25 mmol of diamine compound (CBAPP) was used, and the dianhydride monomer is 1.25 mmol of ODPA.

In addition, the characteristics of PI (PI-4) were analyzed as follows. Relative viscosity: 1.16 in DMAc (solution concentration is 0.5 g dL⁻¹, and the measurement temperature is 30° C.).
Solubility: PI-4 is soluble in NMP and DMAc.
Thermal property: The glass transition temperature is higher than 350° C., the temperature of 10% decomposition is 538° C. in nitrogen, and the temperature of 10% decomposition is 530° C. in air.

### [Embodiment 5] Synthesis of PI (CBAPP-m-DAN) (PI-5)

PI (PI-5) was synthesized in the same manner as that of Embodiment 1, except that 0.99 mmol of diamine compound (CBAPP) was used and the dianhydride monomer was 0.99 mmol of m-DAN.

In addition, the characteristics of PI (PI-5) were analyzed as follows. Relative viscosity: 1.21 in DMAc (solution concentration is 0.5 g dL⁻¹, and the measurement temperature is 30° C.).
Solubility: PI-5 is soluble in NMP, DMAc and pyridine.
Thermal property: The glass transition temperature is higher than 289° C., the temperature of 10% decomposition is 528° C. in nitrogen, and the temperature of 10% decomposition is 521° C. in air.
Thin film mechanical property: The tensile strength is 73.0 MPa, the elongation is 20%, and the tensile coefficient is 1.87 GPa.

### [Embodiment 6] Synthesis of PI (CBAPP-BPDA) (PI-6)

PI (PI-6) was synthesized in the same manner as that of Embodiment 1, except that 0.99 mmol of diamine compound (CBAPP) was used, and the dianhydride monomer was 0.99 mmol of BPDA.

In addition, the characteristics of PI (PI-6) were analyzed as follows. Thermal property: The glass transition temperature is higher than 350° C., the temperature of 10% decomposition is 485° C. in nitrogen, and the temperature of 10% decomposition is 537° C. in air.

### [Embodiment 7] Synthesis of PI (CBAPP-PMDA)(PI-7)

PI (PI-7) was synthesized in the same manner as that of Embodiment 1, except that 0.86 mmol of diamine compound (CBAPP) was used, and the dianhydride monomer was 0.86 mmol of PMDA.

In addition, the characteristics of PI (PI-7) are analyzed as follows. Thermal property: The glass transition temperature is higher than 350° C., the temperature of 10% decomposition is 433° C. in nitrogen, and the temperature of 10% decomposition is 417° C. in air.

### [Embodiment 8] Synthesis of PI Copolymer (Co-PI-1)

0.441 mmol of diamine compound (CBAPP) and 0.441 mmol of hexafluoro diamine monomer represented by formula (8-4) were dissolved in 3.2 mL of N-methyl-2-pyrrolidinone (NMP) solvent. After the dissolution was complete, 0.441 mmol of 6FDA and cyclobutane dianhydride monomers were gradually added therein and reacted for 4 hr, so as to obtain a viscous poly(amic acid) solution. Afterwards, a chemical cyclodehydration process was carried out by adding 0.8 ML of acetic anhydride and 0.4 mL of pyridine into the poly(amic acid) solution at room temperature and stirring the mixture for 1 hr. The reaction mixture was then heated to a temperature lower than 100° C. and stirred for another 3 hr. After the reaction was completed and the mixture was cooled, the NMP solution was poured into a great amount of methanol for precipitation. The precipitate polymer was rinsed by methanol and dried in vacuo at 100° C. to obtain a PI copolymer Co-PI-1.

In addition, the chemical structure of the PI copolymer Co-PI-1 was confirmed and the characteristics thereof were analyzed as follows.
Relative viscosity: 1.2 in N,N-dimethylacetamide (DMAc) (solution concentration is 0.5 g dL⁻¹, and the measurement temperature is 30° C.).
Solubility: Co-PI-1 is soluble in N-methyl-2-pyrrolidinone (NMP), pyridine, THF, N,N-dimethyl acetamide (DMAc), dimethyl sulfoxide (DMSO), chloroform, and cyclohexanone.
Thermal property: The glass transition temperature is higher than 280° C., the temperature of 10% decomposition is higher than 470° C. in nitrogen, and the temperature of 10% decomposition is about 420° C. in air.
Thin film mechanical property: The tensile strength is about 70.0 MPa, the elongation is about 9%, and the tensile coefficient is about 2.3 GPa.

### [Embodiment 9] Synthesis of PI Copolymer (Co-PI-2)

0.441 mmol of diamine compound (CBAPP) and 0.441 mmol of isobutyl diamine monomer represented by formula (8-1) were dissolved in 3.2 mL of N-methyl-2-pyrrolidinone (NMP) solvent. After the dissolution was complete, 0.441 mmol of 6FDA and cyclopentane dianhydride monomers were gradually added therein and reacted for 4 hr, so as to obtain a viscous poly(amic acid) solution. Afterwards, a chemical cyclodehydration process was carried out by adding 0.8 ML of acetic anhydride and 0.4 mL of pyridine into the poly(amic acid) solution at room temperature and stirring the mixture for 1 hr. The reaction mixture was then heated to a temperature lower than 100° C. and stirred for another 3 hr. After the reaction was completed and the mixture was cooled, the NMP solution was poured into a great amount of methanol for precipitation. The precipitate polymer was rinsed by methanol and dried in vacuo at 100° C. to obtain a PI copolymer Co-PI-2.

In addition, the chemical structure of the PI copolymer Co-PI-2 was confirmed and the characteristics thereof were analyzed as follows.
Relative viscosity: 1.2 in N,N-dimethylacetamide (DMAc) (solution concentration is 0.5 g dL⁻¹, and the measurement temperature is 30° C.).
Solubility: Co-PI-2 is soluble in N-methyl-2-pyrrolidinone (NMP), pyridine, THF, N,N-dimethyl acetamide (DMAc), dimethyl sulfoxide (DMSO), chloroform, and cyclohexanone.
Thermal property: The glass transition temperature is higher than 270° C., the temperature of 10% decomposition is higher than 440° C. in nitrogen, and the temperature of 10% decomposition is about 390° C. in air.
Thin film mechanical property: The tensile strength is about 68.0 MPa, the elongation is about 8%, and the tensile coefficient is about 2.2 GPa.

### [Embodiment 10] Synthesis of PI Copolymer (Co-PI-3)

0.441 mmol of diamine compound (CBAPP) and 0.441 mmol of hexamethylene diamine monomer represented by formula (7) in which n=6 were dissolved in 3.6 mL of N-methyl-2-pyrrolidinone (NMP) solvent. After the dissolution was complete, 0.441 mmol of 6FDA and cyclobutane dianhydride monomers were gradually added therein and reacted for 4 hr, so as to obtain a viscous poly(amic acid) solution. Afterwards, a chemical cyclodehydration process was carried out by adding 0.8 mL of acetic anhydride and 0.4 mL of pyridine into the poly(amic acid) solution at room temperature and stirring the mixture for 1 hr. The reaction mixture was then heated to a temperature lower than 100° C. and stirred for another 3 hr. After the reaction was completed and the mixture was cooled, the NMP solution was poured into a great amount of methanol for precipitation. The precipitate polymer was rinsed by methanol and dried in vacuo at 100° C. to obtain a PI copolymer Co-PI-3.

In addition, the chemical structure of the PI copolymer Co-PI-3 was confirmed and the characteristics thereof were analyzed as follows.
Relative viscosity: 1.0 in N,N-dimethylacetamide (DMAc) (solution concentration is 0.5 g dL⁻¹, and the measurement temperature is 30° C.).
Solubility: Co-PI-3 is soluble in N-methyl-2-pyrrolidinone (NMP), pyridine, THF, N,N-dimethyl acetamide (DMAc), dimethyl sulfoxide (DMSO), chloroform, and cyclohexanone.
Thermal property: The glass transition temperature is higher than 280° C., the temperature of 10% decomposition is higher than 450° C. in nitrogen, and the temperature of 10% decomposition is about 400° C. in air.
Thin film mechanical property: The tensile strength is about 64.0 MPa, the elongation is about 7%, and the tensile coefficient is about 2.1 GPa.

### [Embodiment 11] Synthesis of PI Copolymer (Co-PI)

The diamine compound (CBAPP), the diamine monomer represented by formula (7) and the diamine monomer represented by one of formulas (8-1) to (8-7) in various ratios were conducted a polycondensation reaction with 6FDA, Rica acid dianhydride, cyclopentane dianhydride and cyclobutane dianhydride in various ratios. The ratio of diamine compounds, i.e. (CBAPP):diamine monomer represented by formula (7):diamine monomer represented by one of formulas (8-1) to (8-7), ranges between 1:1:1 and 1:5:5, such that a PI copolymer Co-PI can be obtained which is prone to form thin films with desirable mechanical properties.

In addition, the chemical structure of the PI copolymer Co-PI was confirmed and the characteristics thereof were analyzed as follows.
Relative viscosity: greater than 1.0 in N,N-dimethylacetamide (DMAc) (solution concentration is 0.5 g dL⁻¹, and the measurement temperature is 30° C.).
Solubility: Co-PI is soluble in N-methyl-2-pyrrolidinone (NMP), pyridine, THF, N,N-dimethyl acetamide (DMAc), dimethyl sulfoxide (DMSO), chloroform, and cyclohexanone.
Thermal property: The glass transition temperature is higher than 250° C., the temperature of 10% decomposition is higher than 430° C. in nitrogen, and the temperature of 10% decomposition is higher than 380° C. in air.
Thin film mechanical property: The tensile strength is higher than 60.0 MPa, the elongation is higher than 6%, and the tensile coefficient is higher than 2.0 GPa.

In view of the above, the present invention provides a new dinitro compound, and a new diamine compound can be derived from the dinitro compound, in which the new diamine compound has a pyridine heterocyclic group and a carbazole-containing electrochromic group. Further, a new PI and new PI copolymers can be fabricated with the new diamine compound and dianhydride, it can be known from the above embodiments that the new PI has good physical properties. Particularly, the new PI has good solubility, and has good solubility in high polarity organic solvents (for example, NMP and DMAc), and most of the new PI is dissolved in low polarity organic solvent (for example, pyridine and THF). Therefore, the processibility of PI is improved, thus the application is increased. The thermal endurance is extremely high when thermal cyclodehydration process is used, so as to directly get the product.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

What is claimed is:

1. A 4-(9-ethyl-3-carbazole)-2,6-bis(4-nitrophenyl)pyridine (CBNPP) dinitro compound, shown in formula (1):

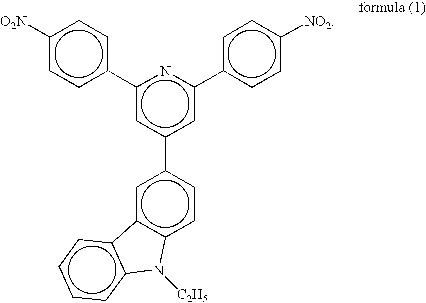

2. A 4-(9-ethyl-3-carbazole)-2,6-bis(4-aminophenyl)pyridine (CBAPP) diamine compound, fabricated with the dinitro compound according to claim 1 as monomer, and shown in formula (2):

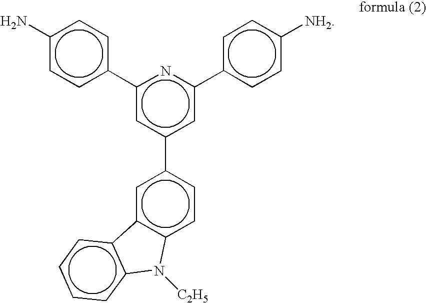

3. A polyimide (PI), fabricated by performing a polycondensation reaction with diamine compound according to claim 2 and a dianhydride shown in formula (3) as monomers, and shown in formula (4): 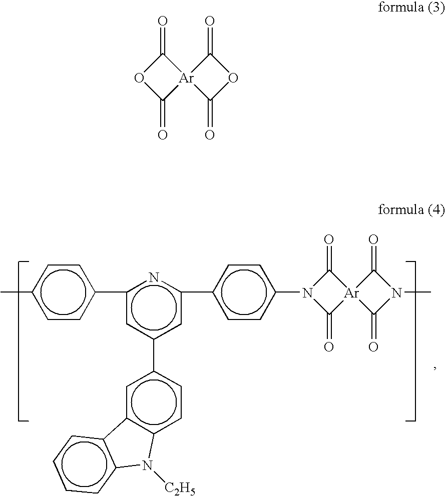
wherein Ar in formula (3) and formula (4) represents aromatic groups.

4. The PI according to claim 3, wherein Ar is a group shown in formula (5-1):

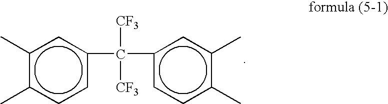

5. The PI according to claim 3, wherein Ar is a group shown in formula (5-2):

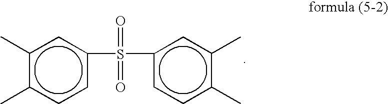

6. The PI according to claim 3, wherein Ar is a group shown in formula (5-3):

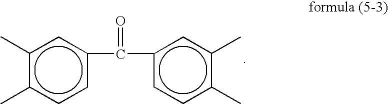

7. The PI according to claim 3, wherein Ar is a group shown in formula (5-4):

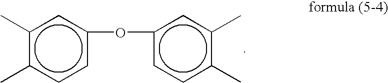

8. The PI according to claim 3, wherein Ar is a group shown in formula (5-5):

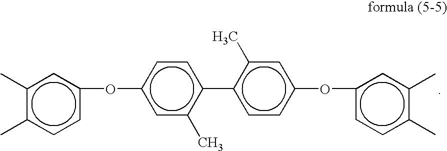

9. The PI according to claim 3, wherein Ar is a group shown in formula (5-6):

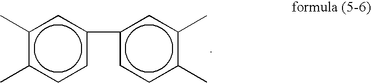

10. The PI according to claim 3, wherein Ar is a group shown in formula (5-7):

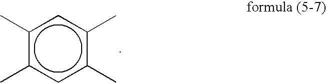

11. A polyimide (PI) copolymer, fabricated by performing a polycondensation reaction using the diamine compound according to claim 2, a diamine compound shown in formulas (7), (8-1), (8-2), (8-3), (8-4), (8-5), (8-6) and (8-7) and a dianhydride compound shown in formula (6) as monomers: 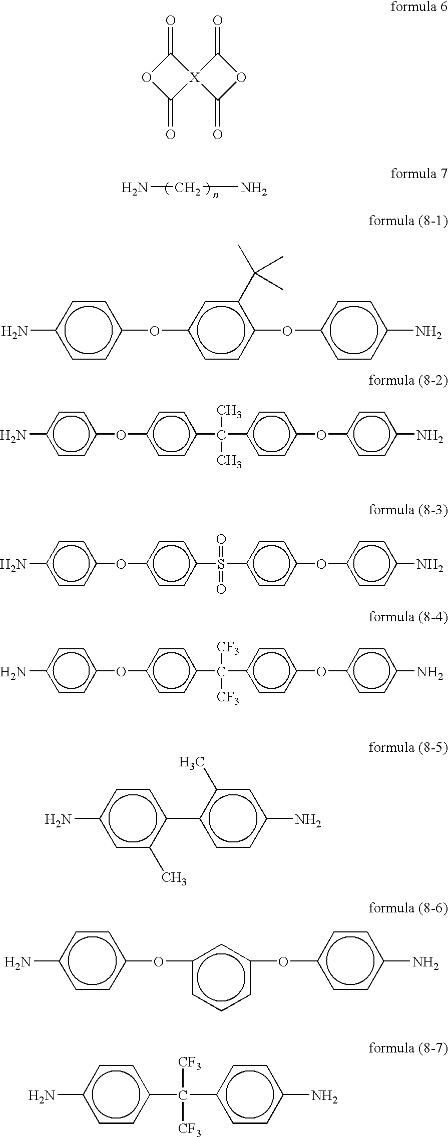
wherein X in formula (6) represents aromatic groups or alicyclic groups, and n=1 to 10 in formula (7).

12. The PI copolymer according to claim 11, wherein X is a group shown in formula (6-1):

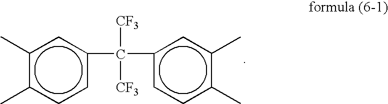

13. The PI copolymer according to claim 11, wherein X is a group shown in formula (6-2):

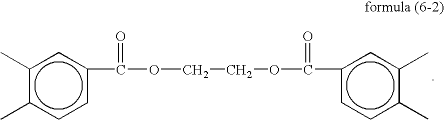

14. The PI copolymer according to claim 11, wherein X is a group shown in formula (6-3):

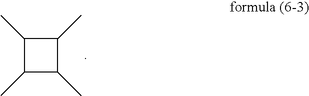

15. The PI copolymer according to claim 11, wherein X is a group shown in formula (6-4):

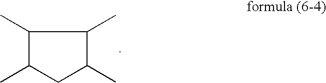

16. The PI copolymer according to claim 11, wherein n=2 or 6 in formula (7).
